(19) United States
(12) Patent Application Publication
(10) Pub. No.: US 2025/0339072 A1
(43) Pub. Date: Nov. 6, 2025

(54) VEHICULAR INTERIOR REARVIEW MIRROR ASSEMBLY WITH DRIVER MONITORING MODULE

(71) Applicant: Magna Mirrors of America, Inc., Holland, MI (US)

(72) Inventors: Nathan J. Pastoor, Jenison, MI (US); Javier Garcia Mata, Barcelona (ES)

(21) Appl. No.: 19/194,102

(22) Filed: Apr. 30, 2025

Related U.S. Application Data
(60) Provisional application No. 63/641,557, filed on May 2, 2024.

(51) Int. Cl. A61B 5/18; A61B 5/00; B60N 2/00; B60Q 3/76; B60R 1/04; B60R 1/12; B60R 16/03; G06V 20/52; G06V 20/59; G06V 40/10; H04N 23/56

(52) U.S. Cl. CPC A61B 5/18; A61B 5/0077; A61B 5/6893; B60N 2/0023; B60Q 3/76; B60R 1/04; B60R 1/1207; B60R 16/03; G06V 20/52; G06V 20/597; G06V 40/10; H04N 23/56; B60R 2001/1253

(57) ABSTRACT

A vehicular cabin monitoring system includes an interior rearview mirror assembly having a mirror head adjustable about a mounting structure configured to mount the mirror assembly at an interior portion of an interior cabin of a vehicle. The mirror head accommodates a mirror reflective element. A cabin monitoring module is disposed at least partially within the mirror head and includes a camera that views at least a driver's head region of the interior cabin and a near infrared light emitter that is operable to emit near infrared light at least toward the driver's head region. The cabin monitoring module includes a cover element that is disposed at a cutout region of the mirror reflective element. An electronic control unit (ECU) includes an image processor for processing image data captured by the camera. Image data captured by the camera is processed at the ECU for monitoring the driver of the vehicle.

### CROSS REFERENCE TO RELATED APPLICATION

The present application claims the filing benefits of U.S. provisional application Ser. No. 63/641,557, filed May 2, 2024, which is hereby incorporated herein by reference in its entirety.

### FIELD OF THE INVENTION

The present invention relates generally to interior rearview mirror assemblies and, more particularly, to an interior rearview mirror assembly that includes a camera for cabin monitoring.

### BACKGROUND OF THE INVENTION

It is known to provide a mirror assembly that is adjustably mounted to an interior portion of a vehicle, such as via a single ball pivot or joint mounting configuration or a double ball pivot or joint mounting configuration, where the mirror casing and reflective element are adjusted relative to the interior portion of a vehicle by pivotal movement about the single or double ball pivot configuration. The mirror casing and reflective element are pivotable about either or both of the ball pivot joints by a user that is adjusting his or her rearward view.

### SUMMARY OF THE INVENTION

A vehicular interior rearview mirror assembly includes a mirror head adjustably attached at a mounting structure that is configured to mount the interior rearview mirror assembly at an interior portion of a cabin of a vehicle equipped with a vehicular driver monitoring system. The mirror head accommodates a mirror reflective element. A driver monitoring module is accommodated by the mirror head and moves together and in tandem with the mirror head when, with the mounting structure attached at the interior portion of the cabin of the vehicle, the mirror head is adjusted relative to the mounting structure to set a rearward view of the driver of the vehicle. The driver monitoring module includes a driver monitoring camera that, with the mounting structure attached at the interior portion of the vehicle, and with the mirror head adjusted relative to the mounting structure to set the rearward view of the driver of the vehicle, views at least a driver's head region of the cabin of the vehicle. The driver monitoring module includes a near infrared light emitter that is electrically operable to emit near infrared light, and emits near infrared light at least toward the driver's head region (e.g., the near infrared light emitter may emit a beam of near infrared light that has a principal beam axis angled generally toward the driver side of the vehicle). An electronic control unit (ECU) includes electronic circuitry and associated software, including an image processor for processing image data captured by the driver monitoring camera. The driver monitoring system, based on processing at the ECU of image data captured by the driver monitoring camera, monitors the driver. The driver monitoring module may be snap-attached or fastened (such as, for example, via one or more threaded fasteners) at the mirror head at a lower region or chin region of the mirror head below the mirror reflective element.

These and other objects, advantages, purposes and features of the present invention will become apparent upon review of the following specification in conjunction with the drawings.

### BRIEF DESCRIPTION OF THE DRAWINGS

FIG. 1 is a perspective view of an interior rearview mirror assembly having a driver monitoring camera and light emitter;

FIG. 2 is an exploded perspective view of the interior rearview mirror assembly of FIG. 1;

FIGS. 3-6 are rear views of the mirror assembly, with the mirror casing removed;

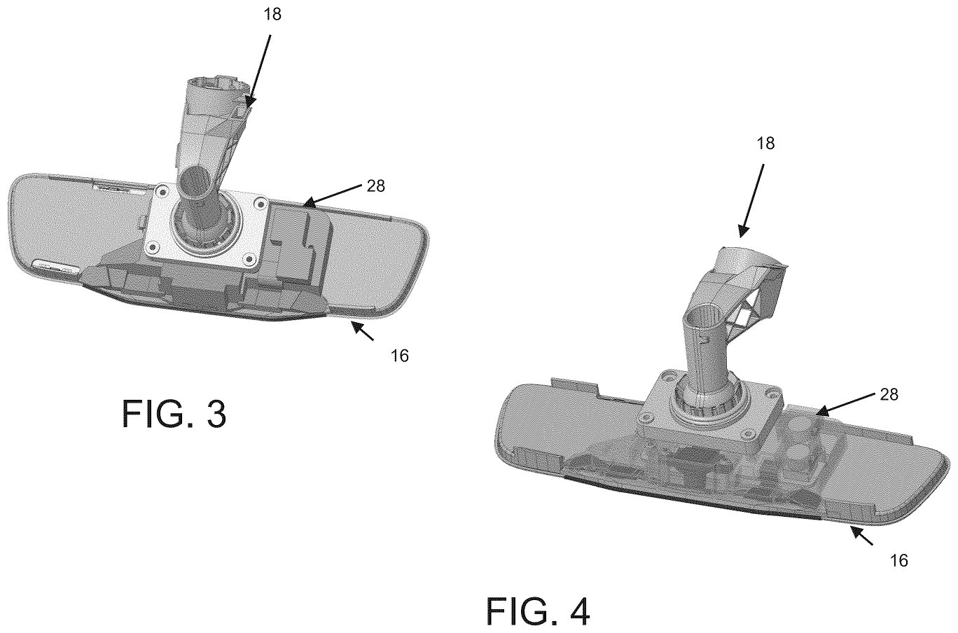

FIG. 7 is a perspective view of the camera and light emitters attached at the attachment plate of the mirror assembly;

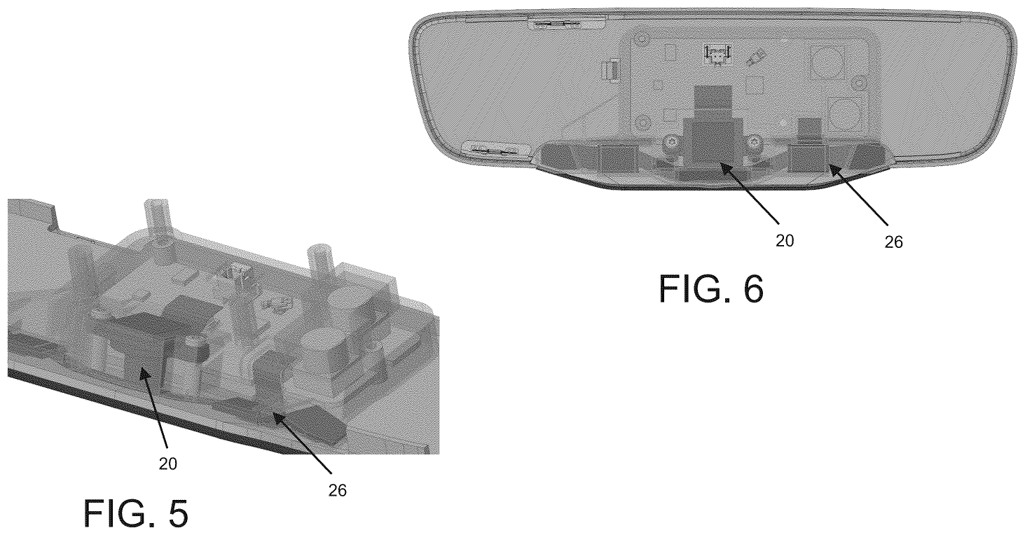

FIG. 8 is an exploded perspective view of a driver monitoring module that is configured to attach at a mirror head of an interior rearview mirror assembly;

FIGS. 9-12 are perspective views of the driver monitoring module of FIG. 8;

FIG. 13 is a view of a mirror head with a driver monitoring module attached at a lower or chin region of the mirror head;

FIG. 14 is an exploded perspective view of the mirror head and driver monitoring module of FIG. 13;

FIGS. 15 and 16 are views of the driver monitoring module attached at the mirror head;

FIG. 17 is a side perspective view of the mirror assembly with the driver monitoring module attached; and

FIG. 18 is a view of the mirror head with the driver monitoring module removed.

### DESCRIPTION OF THE PREFERRED EMBODIMENTS

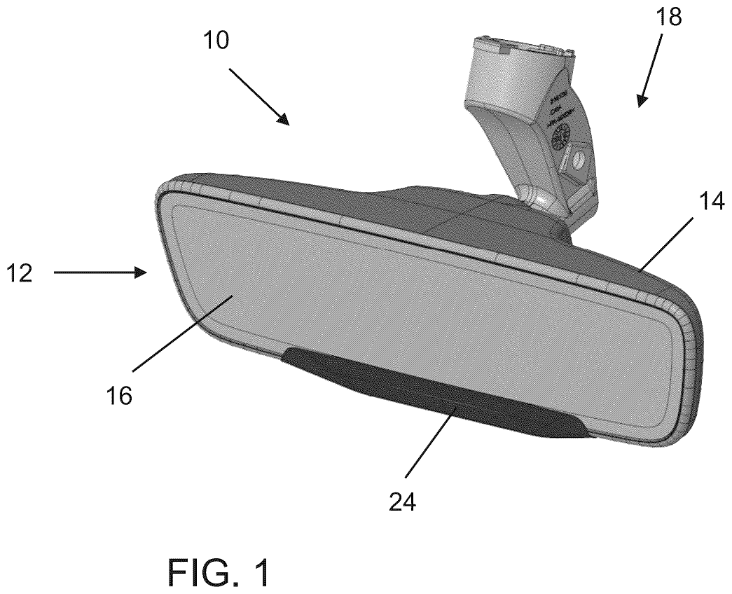

Referring now to the drawings and the illustrative embodiments depicted therein, an interior rearview mirror assembly 10 for a vehicle includes a mirror head 12 that accommodates a mirror casing 14 and a mirror reflective element 16 (FIG. 1). In the illustrated embodiment, the mirror assembly 10 is configured to be adjustably mounted to an interior portion of a vehicle (such as to an interior or in-cabin surface of a vehicle windshield or a headliner of a vehicle or the like) via a mounting structure or mounting configuration or assembly 18. The mirror reflective element may comprise a variable reflectance mirror reflective element, such as an electrochromic mirror reflective element, that varies its reflectance responsive to electrical current applied to conductive coatings or layers of the reflective element.

The mirror assembly 10 includes or is associated with a driver monitoring system (DMS) and/or an occupant monitoring system (OMS), with the mirror assembly including a driver/occupant monitoring camera 20 disposed at a back plate or attachment plate 22 (and viewing through an aperture of the back plate). The camera is disposed at a lower region of the attachment plate 22 and below the mirror reflective element 16 and viewing through a lower cover element 24 toward at least a head region of the driver of the vehicle. The DMS may include an infrared light (IR light) or near infrared light (near IR light) emitter 26 disposed at the back plate and emitting IR light or near IR light that passes through another aperture of the back plate for illuminating at least the head region of the driver of the vehicle with near IR light. Further, the monitoring system includes an electronic control unit (ECU) having electronic circuitry and associated software, including an image processor for processing image data captured by the DMS/OMS camera.

Image data captured by the camera may be processed for a head and face direction and position tracking system and/or eye tracking system and/or gesture recognition system. The DMS camera and monitoring system and/or head and face direction and/or position tracking systems and/or eye tracking systems and/or gesture recognition systems may utilize aspects of the systems described in U.S. Pat. Nos. 11,827,153; 11,780,372; 11,639,134; 11,582,425; 11,518,401; 10,958,830; 10,065,574; 10,017,114; 9,405,120 and/or 7,914, 187, and/or U.S. Publication Nos. US-2024-0383406; US-2024-0190456; US-2024-0168355; US-2022-0377219; US-2022-0254132; US-2022-0242438; US-2021-0323473; US-2021-0291739; US-2020-0320320; US-2020-0202151; US-2020-0143560; US-2019-0210615; US-2018-0231976; US-2018-0222414; US-2017-0274906; US-2017-0217367; US-2016-0209647; US-2016-0137126; US-2015-0352953; US-2015-0296135; US-2015-0294169; US-2015-0232030; US-2015-0092042; US-2015-0022664; US-2015-0015710; US-2015-0009010 and/or US-2014-0336876, and/or U.S. provisional application Ser. No. 63/673,225, filed Jul. 19, 2024, and/or U.S. provisional application Ser. No. 63/641,574, filed May 2, 2024, and/or International Publication No. WO 2023/220222, which are all hereby incorporated herein by reference in their entireties.

With the DMS camera 20 disposed at the mirror head 12, the camera 20 moves together and in tandem with the mirror head 12 (including the mirror casing 14 and mirror reflective element 16 that pivot at a pivot joint that pivotally connects the mirror head to the mounting structure 18 of the interior rearview mirror assembly that in turn mounts at a windshield or at a headliner of the equipped vehicle), such that, when the driver aligns the mirror head 12 to view rearward, the camera 20 is positioned so as to view at least the driver of the vehicle. The location of the DMS camera 20 and IR LED(s) 26 at the mirror head 12 provide an unobstructed view to the driver (through the cover element 24). The driver monitoring camera 20 may also provide captured image data for a cabin monitoring system or occupancy monitoring system (OMS) or another separate OMS camera may be disposed at the mirror assembly for the OMS function.

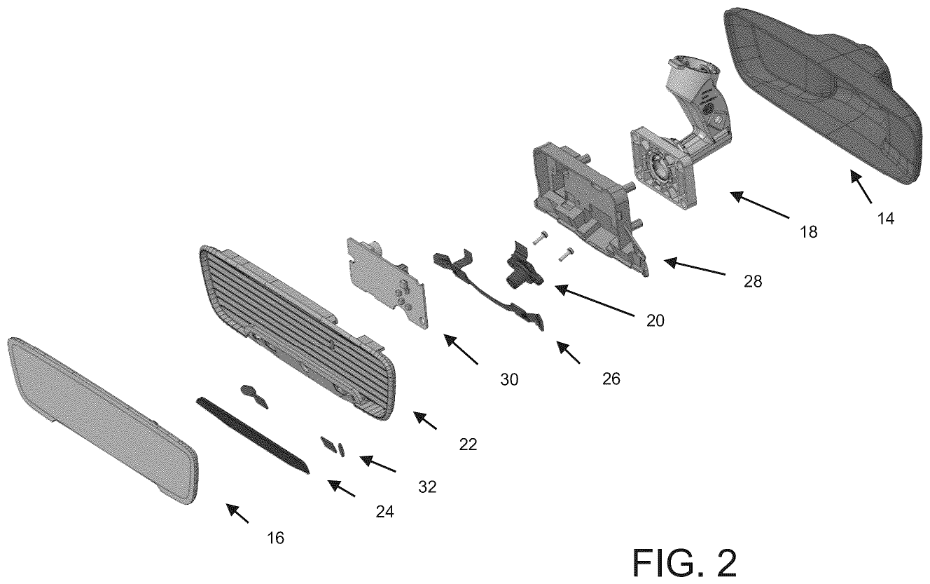

As shown in FIG. 2, the camera 20 and near IR light emitters 26 are disposed at the attachment plate 22 so as to respectively view through and emit light through apertures in the attachment plate and through the cover element 24, which is disposed at a lower region or cutout region of the mirror reflective element 16. That is, the camera 20 is disposed at the attachment plate 22 and views through an aperture in the attachment plate and the near IR light emitters 26 are disposed at the attachment plate 22 spaced from the camera 20 and emit light that passes through respective apertures in the attachment plate 22. The mirror assembly includes a heat sink 28 that dissipates heat generated by the camera and light emitters when operating. The camera and light emitters include respective electrical connectors that are electrically connected to a serializer board 30 for controlling the camera and light emitters and for receiving image data captured by the camera. The mirror may also include red glow filters 32 disposed at the apertures of the attachment plate that align with the light emitters 26. Thus, light emitted by the near IR light emitters 26 may pass through the red glow filters 32 and illuminate a portion of the interior cabin of the vehicle.

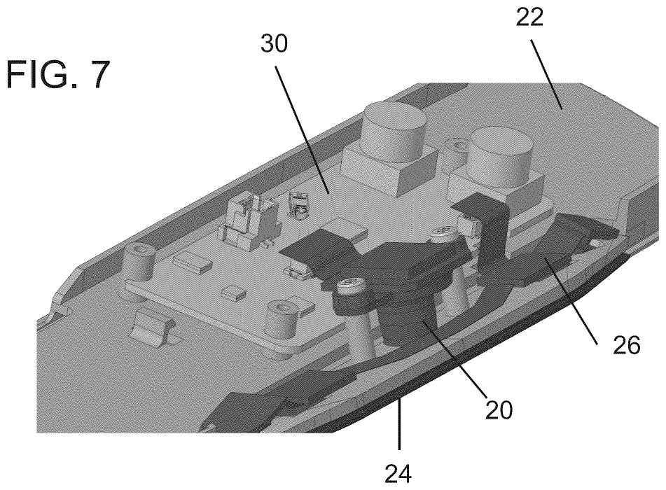

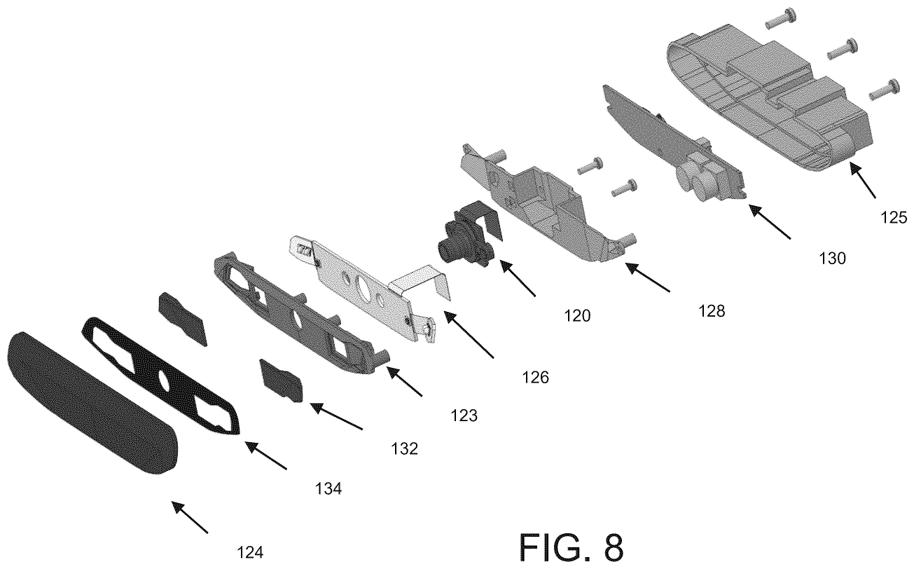

Optionally, the driver/occupant monitoring camera and/or one or more near IR light emitters may comprise part of a cabin monitoring module or driver monitoring module that is attachable as a unit at the mirror head, such as for ease of assembly and simpler maintenance. With reference to FIGS. 8-12, the mirror assembly may accommodate a driver or occupant monitoring module or cabin monitoring module 111 that is attachable (such as via snap attachment and/or via fasteners) at the mirror head to integrate a driver monitoring system into the mirror assembly. As shown in FIG. 8, the driver monitoring module 111 includes a housing or heat sink 125 and a cover element 124, which encase or house a camera 120 and light emitters (disposed at a light emitter printed circuit board (PCB) 126). In other words, the housing 125 and the cover element 124 join together to house or accommodate the camera 120 and the PCB 126. The light emitters and/or the camera 120 may be disposed at the PCB 126. Optionally, the camera 120 is disposed at or includes a separate imager PCB.

The module includes a lens frame 123 that attaches at the cover element 124, with the light emitter PCB 126 and camera 120 attached at the lens frame or at a light emitter heat sink 128. The housing 125 attaches at the lens frame 123 to house or encase the heat sink and camera and light emitters and a serializer board 130 within the module. The cover element 124 may be adhesively attached at the lens frame 123 (such as via tape 134 or other suitable adhesive), and one or more red glow filters 132 may be disposed at the lens frame in front of the light emitters of the light emitter PCB. The camera 120 and the light emitter PCB 126 each include an electrical connector (such as a flexible circuit element or the like) that is routed around or through the heat sink 128 and electrically connected to circuitry at the serializer board 130, which includes an electrical connector 136 at a rear side of the board.

Thus, the driver monitoring module provides a unitary self-contained module that includes the driver monitoring camera and/or occupant monitoring camera and light emitters and associated circuitry. When installed at a mirror head, the driver monitoring module is attached at the mirror head and the electrical connector 136 (see FIG. 11) of the circuitry is accessible for electrical connection to an electrical connector at the mirror head.

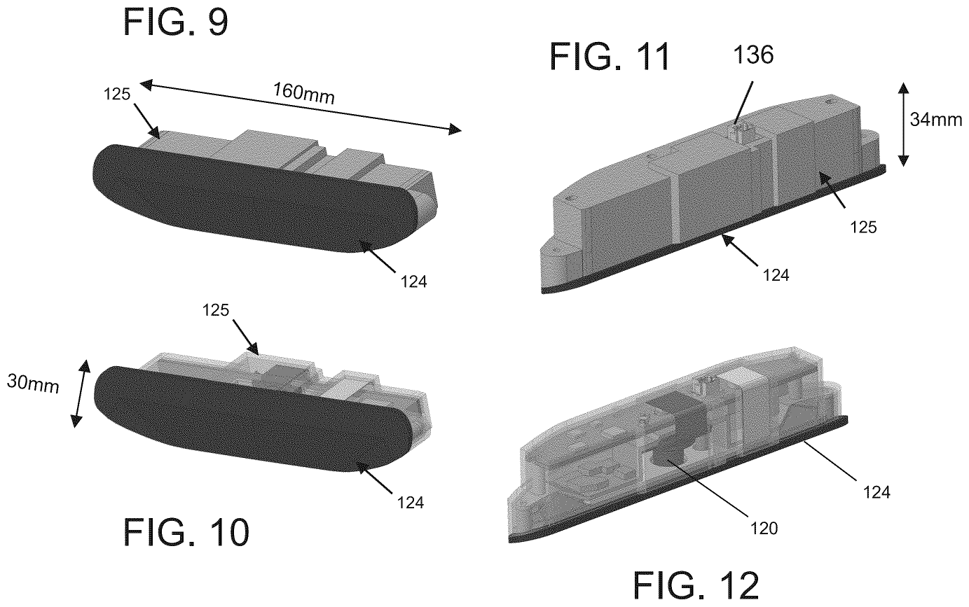

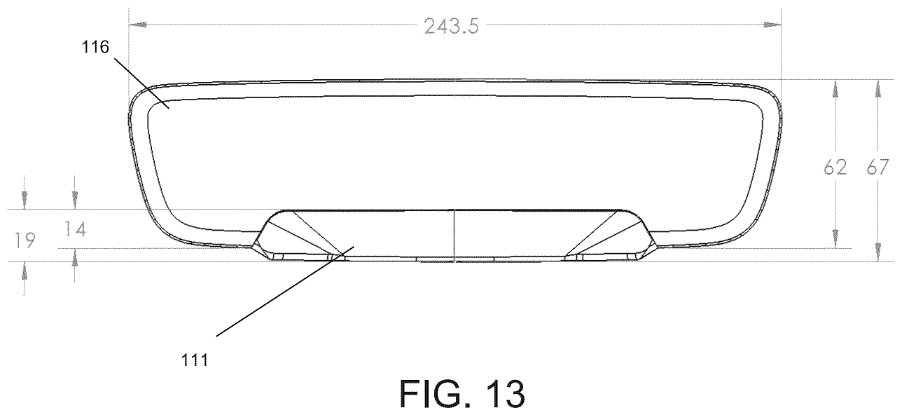

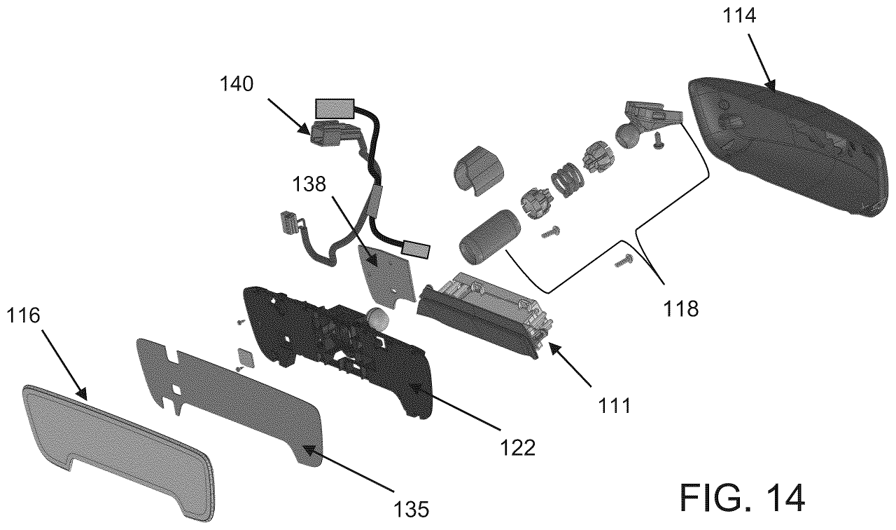

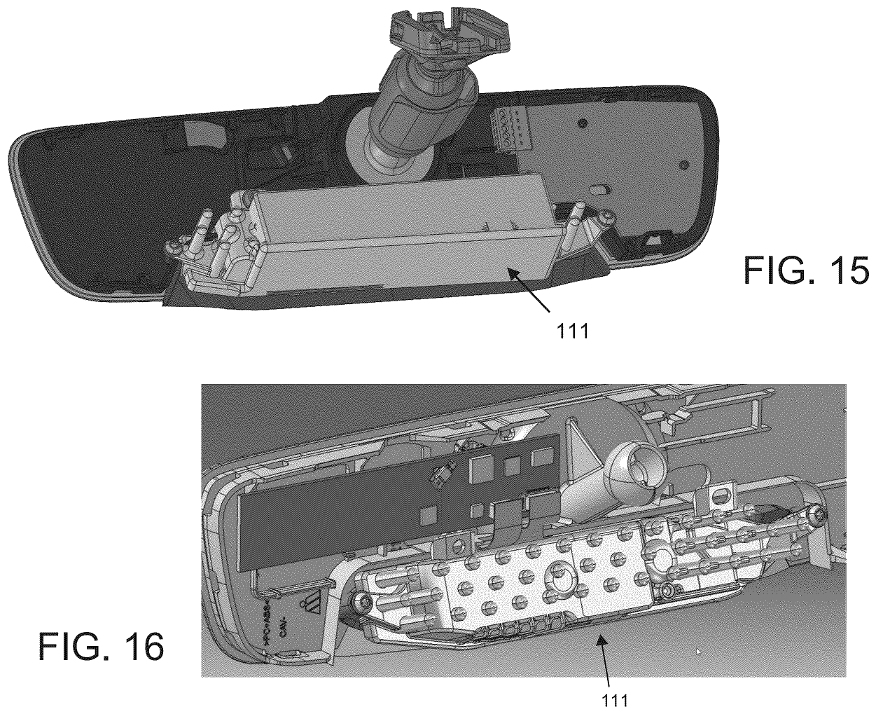

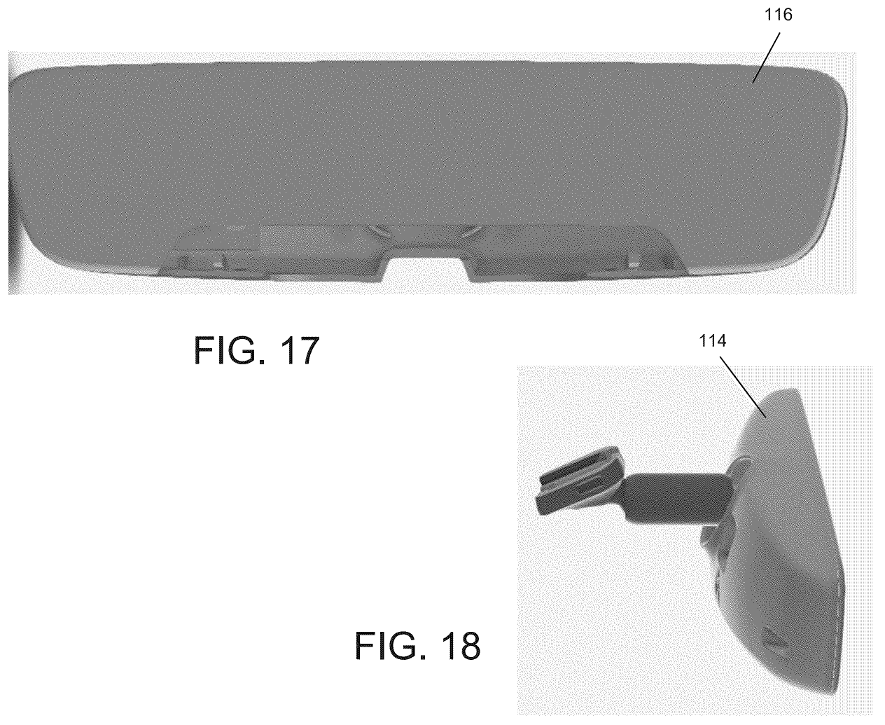

For example, and such as shown in FIGS. 13-18, the module 111 may connect to a cutout region of a mirror head 112, such that the cover element 124 of the module is located at a cutout region of the mirror reflective element 116. The cutout region may comprise a recess or notch at a lower edge region of the mirror reflective element 116. As shown in FIG. 14, the module 111 attaches at the attachment plate 122, which is adhesively attached (such as via tape 135 or other suitable adhesive) at the rear side of the mirror reflective element 116 and also attaches at the mirror casing 114 (such as via snap attachment or threaded fasteners). The module 111 may attach to the attachment plate 122 via tabs that are received by respective receiving portions at the attachment plate 122. An outer surface of the cover 124 of the module 111 may be substantially parallel to and flush or coplanar with an outer surface of the mirror reflective element 116 at the lower edge region. An imager plane of the camera 120 may be parallel to the outer surface of the cover 124. Optionally, the cover 124 (and thus the imager plane of the camera 120) may be disposed at an oblique angle relative to the mirror reflective element 116, such as to accommodate a viewing angle of the camera 120 relative to a driver region and/or passenger seating region within the interior cabin of the vehicle. For example, the imager plane and the cover 124 may be disposed at an angle of about 1 degree or less, about 5 degrees or less, about 10 degrees or less, about 15 degrees or less, and the like.

The mirror head may include a circuit board 138 that includes EC dimming circuitry (for applications where the mirror reflective element is a variable reflectance auto-dimming mirror). The mirror assembly includes a wiring harnesses 140 for electrically connecting the driver monitoring module and the EC dimming circuitry to a wire harness of the vehicle when the mirror assembly is mounted at an interior portion of the vehicle via the mounting structure 118.

The near IR light emitters of the driver monitoring module may comprise one or more infrared (IR) light emitters (such as IR or near-IR light emitting diodes (LEDs) or vertical-cavity surface-emitting lasers (VCSEL) or the like) disposed at the back plate and emitting near infrared light through the aperture of the back plate and through the cover element toward the head region of the driver of the vehicle. The light emitter printed circuit board may have a first set of near infrared light emitting diodes (e.g., a set of wider beam LEDs) at one part of the PCB and a second set of near infrared light emitting diodes (e.g., a set of narrower beam LEDs) at another part of the PCB. The PCB may have one part angled relative to the other part to emit light in a desired direction depending on the orientation of the mirror head. Thus, the first set of near infrared light emitting diodes may be angled toward the left side of the vehicle so as to be directed toward a driver of a left hand drive vehicle (if the mirror assembly is installed in a left hand drive vehicle and the first set of near infrared light emitting diodes are enabled for the driver monitoring function), while the second set of near infrared light emitting diodes may be angled toward the right side of the vehicle so as to be directed toward a driver of a right hand drive vehicle (if the mirror assembly is installed in a right hand drive vehicle and the second set of near infrared light emitting diodes are enabled for the driver monitoring function).

The driver monitoring module thus may have an embedded camera, near IR illuminators and a processor for processing captured image data for the driver monitoring application. The inward facing camera and near IR illuminators are fixed within the module and, thus, when the module is fixedly attached at the mirror head, are also fixed at the mirror head, and thus both components are coupled with the mirror body.

Optionally, the mirror back plate or attachment plate may be molded out of a metal filled injection moldable material (e.g., Stainless Steel (SS) fiber, such as a polycarbonate (PC) Acrylonitrile butadiene styrene (ABS) and SS fiber material) to provide electromagnetic interference (EMI) mitigation (EMC shield). Optionally, the heatsink may be formed via additive manufacturing (3D printing or the like) to provide an additive manufactured heatsink with capillary effect to help transfer heat more uniformly and away from high power components.

The filters enhance covertness of the system by limiting visible light to avoid any visible light that may be emitted by the near-IR light emitters from being visible through the mirror cover element (and thus reduces or avoids LED red glow being visible at the cover element when the LEDs are powered). The filters also block or limit incursion of ambient cabin light into the mirror head at the locations where the camera views through the cover element to see into the vehicle cabin.

Thus, the driver monitoring module provides a self-contained module that includes the driver monitoring camera and the near infrared light emitters. The module is attached at the mirror head and electrically connected to a wire harness within the mirror head. With the driver monitoring module attached at the mirror head, the camera and light emitters are disposed at the chin region or lower region of the mirror head, with the cover element disposed immediately below the mirror reflective element, and optionally at a recessed or cutout region of the mirror reflective element. The driver monitoring module is thus accommodated by the mirror head below the mirror reflective element, and the driver monitoring camera views through the cover element and not through the mirror reflective element and the near infrared light emitter emits near infrared light through the cover element and not through the mirror reflective element.

The driver monitoring module and constructions can be utilized with other modules integrated into the interior rearview mirror assembly, such as by utilizing aspects of the garage door opener modules and systems described in U.S. Pat. Nos. 11,046,251; 10,864,865 and/or 10,189,411, which are hereby incorporated herein by reference in their entireties.

Optionally, the DMS camera may be used to detect ambient light and/or glare light (emanating from headlamps of a trailing vehicle) for use in providing auto-dimming of the EC mirror reflective element. The DMS camera may be disposed in the mirror head and viewing rearward through the mirror reflective element. The processing of image data captured by the DMS camera may be adjusted to accommodate the angle of the mirror head so that the ECU or system, via image processing of image data captured by the DMS camera, determines headlamps of a trailing vehicle (behind the equipped vehicle and traveling in the same direction as the equipped vehicle and traveling in the same traffic lane or in an adjacent traffic lane) to determine glare light at the mirror reflective element. The processing of image data captured by the DMS camera is adjusted to accommodate the degree of dimming of the mirror reflective element. For example, the system knows how much the mirror reflective element is dimmed (responsive to the determined glare light intensity and location) and can accommodate for the mirror dimming level when processing captured image data to determine presence and intensity of light sources/headlamps rearward of the vehicle. The intelligent/automatic mirror dimming functions may utilize aspects of the systems described in U.S. Pat. Nos. 11,780,372; 11,242,008; 10,967,796 and/or 10,948,798, and/or U.S. Publication No. US-2024-0064274, which are all hereby incorporated herein by reference in their entireties.

The interior mirror assembly may comprise a dual-mode interior rearview video mirror that can switch from a traditional reflection mode to a live-video display mode, such as by utilizing aspects of the mirror assemblies and systems described in U.S. Pat. Nos. 11,242,008; 11,214,199; 10,442,360; 10,421,404; 10,166,924; 10,046,706 and/or 10,029,614, and/or U.S. Publication Nos. US-2021-0162926; US-2021-0155167; US-2020-0377022; US-2019-0258131; US-2019-0146297; US-2019-0118717 and/or US-2017-0355312, which are all hereby incorporated herein by reference in their entireties. The video display screen of the video mirror, when the mirror is in the display mode, may display video images derived from video image data captured by a rearward viewing camera, such as a rearward camera disposed at a center high-mounted stop lamp (CHMSL) location, and/or video image data captured by one or more other cameras at the vehicle, such as side-mounted rearward viewing cameras or the like, such as by utilizing aspects of the display systems described in U.S. Pat. No. 11,242,008, which is hereby incorporated herein by reference in its entirety. The operating mode of the mirror and video display screen may be selected by flipping the mirror head upward or downward (e.g., via a toggle located at the mirror head) or responsive to another user input. When the mirror is operating in the mirror mode, the video display screen is deactivated and rendered covert by the mirror reflective element, and the driver views rearward via reflection of light incident at the mirror reflective element. When the mirror is operating in the display mode, the video display screen is operated to display video images that are viewable through the mirror reflective element by the driver of the vehicle.

The mirror assembly may comprise any suitable construction, such as, for example, a mirror assembly with the reflective element being nested in the mirror casing and with a bezel portion that circumscribes a perimeter region of the front surface of the reflective element, or with the mirror casing having a curved or beveled outermost exposed perimeter edge around the reflective element and with no overlap onto the front surface of the reflective element (such as by utilizing aspects of the mirror assemblies described in U.S. Pat. Nos. 7,184,190; 7,274,501; 7,255,451; 7,289,037; 7,360,932; 7,626,749; 8,049,640; 8,277,059 and/or 8,529,108, which are hereby incorporated herein by reference in their entireties) or such as a mirror assembly having a rear substrate of an electro-optic or electrochromic reflective element nested in the mirror casing, and with the front substrate having a curved or beveled outermost exposed perimeter edge, or such as a mirror assembly having a prismatic reflective element that is disposed at an outer perimeter edge of the mirror casing and with the prismatic substrate having a curved or beveled outermost exposed perimeter edge, such as described in U.S. Pat. Nos. 9,827,913; 9,174,578; 8,508,831; 8,730,553; 9,598,016 and/or 9,346,403, and/or U.S. Des. Pat. Nos. D633,423; D633,019; D638,761 and/or D647,017, which are hereby incorporated herein by reference in their entireties (and with electrochromic and prismatic mirrors of such construction are commercially available from the assignee of this application under the trade name INFINITY™ mirror).

As discussed above, the mirror assembly may comprise an electro-optic or electrochromic mirror assembly that includes an electro-optic or electrochromic reflective element. The perimeter edges of the reflective element may be encased or encompassed by the perimeter element or portion of the bezel portion to conceal and contain and envelop the perimeter edges of the substrates and the perimeter seal disposed therebetween. The electrochromic mirror element of the electrochromic mirror assembly may utilize the principles disclosed in commonly assigned U.S. Pat. Nos. 7,274,501; 7,255,451; 7,195,381; 7,184,190; 6,690,268; 5,140,455; 5,151,816; 6,178,034; 6,154,306; 6,002,544; 5,567,360; 5,525,264; 5,610,756; 5,406,414; 5,253,109; 5,076,673; 5,073,012; 5,117,346; 5,724,187; 5,668,663; 5,910,854; 5,142,407 and/or 4,712,879, which are hereby incorporated herein by reference in their entireties.

Optionally, the driver monitoring module and/or the mirror assembly may utilize aspects of the accessory modules and/or the mirror assemblies described in U.S. Pat. Nos. 10,189,411; 10,864,865 and/or 11,046,251, which are hereby incorporated herein by reference in their entireties.

Changes and modifications in the specifically described embodiments may be carried out without departing from the principles of the present invention, which is intended to be limited only by the scope of the appended claims as interpreted according to the principles of patent law.

1. A vehicular cabin monitoring system, the vehicular cabin monitoring system comprising:
a vehicular interior rearview mirror assembly comprising a mirror head adjustable about a mounting structure, wherein the mounting structure is configured to mount the vehicular interior rearview mirror assembly at an interior portion of an interior cabin of a vehicle;
wherein the mirror head accommodates a mirror reflective element;
a cabin monitoring module attached at the mirror head, wherein the cabin monitoring module moves together and in tandem with the mirror head when, with the vehicular interior rearview mirror assembly mounted at the interior portion of the interior cabin of the vehicle, the mirror head is adjusted about the mounting structure to set a rearward view provided by the mirror reflective element for a driver of the vehicle;
wherein the cabin monitoring module comprises a housing that is disposed at least partially within the mirror head, and wherein the housing accommodates a camera, and wherein, with the vehicular interior rearview mirror assembly mounted at the interior portion of the interior cabin of the vehicle, and with the mirror head adjusted about the mounting structure to set the rearward view for the driver of the vehicle, the camera views at least a driver's head region of the interior cabin of the vehicle;
wherein the housing of the cabin monitoring module accommodates a near infrared light emitter that is electrically operable to emit near infrared light, and wherein the near infrared light emitter, when electrically operated to emit near infrared light, and with the vehicular interior rearview mirror assembly mounted at the interior portion of the vehicle, and with the mirror head adjusted about the mounting structure to set the rearward view for the driver of the vehicle, emits near infrared light at least toward the driver's head region;
wherein the cabin monitoring module includes a cover element attached at the housing, and wherein, with the cabin monitoring module attached at the mirror head, the cover element is disposed at a cutout region of the mirror reflective element;
an electronic control unit (ECU);
wherein image data captured by the camera is transferred to the ECU;
wherein the ECU comprises electronic circuitry and associated software, and wherein the electronic circuitry of the ECU comprises an image processor operable to process image data captured by the camera and transferred to the ECU; and
wherein, with the vehicular interior rearview mirror assembly mounted at the interior portion of the interior cabin of the vehicle, image data captured by the camera is processed at the ECU for monitoring the driver of the vehicle.

2. The vehicular cabin monitoring system of claim 1, wherein image data captured by the camera is processed at the ECU for at least one selected from the group consisting of (i) detecting a passenger of the vehicle and (ii) monitoring a passenger of the vehicle.

3. The vehicular cabin monitoring system of claim 1, wherein the near infrared light emitter, when electrically operated to emit near infrared light, and with the mirror head adjusted about the mounting structure to set the rearward view for the driver of the vehicle, emits near infrared light to illuminate a passenger seating region of the vehicle.

4. The vehicular cabin monitoring system of claim 1, wherein a driver gaze direction is determined based on processing at the ECU of image data captured by the camera.

5. The vehicular cabin monitoring system of claim 1, wherein driver attentiveness is determined based on processing at the ECU of image data captured by the camera.

6. The vehicular cabin monitoring system of claim 1, wherein driver drowsiness is determined based on processing at the ECU of image data captured by the camera.

7. The vehicular cabin monitoring system of claim 1, wherein driver distraction is determined based on processing at the ECU of image data captured by the camera.

8. The vehicular cabin monitoring system of claim 1, wherein the housing of the cabin monitoring module accommodates the ECU.

9. The vehicular cabin monitoring system of claim 1, wherein the mirror head accommodates the ECU separate from the cabin monitoring module.

10. The vehicular cabin monitoring system of claim 1, wherein the ECU is disposed within the vehicle remote from the vehicular interior rearview mirror assembly.

11. The vehicular cabin monitoring system of claim 1, wherein the cabin monitoring module includes a heat sink.

12. The vehicular cabin monitoring system of claim 1, wherein the cabin monitoring module comprises a self-contained unit that is attached at the mirror head and that electrically connects to a wire harness at the mirror head for connection to a power source of the vehicle when the vehicular interior rearview mirror assembly is mounted at the interior portion of the interior cabin of the vehicle.

13. The vehicular cabin monitoring system of claim 1, wherein the housing of the cabin monitoring module is configured to attach at a mirror back plate that adhesively attaches at the mirror reflective element.

14. The vehicular cabin monitoring system of claim 13, wherein the mirror back plate has a cutout region to accommodate the cabin monitoring module, and wherein the cutout region of the mirror back plate corresponds with the cutout region of the mirror reflective element.

15. The vehicular cabin monitoring system of claim 14, wherein the housing of the cabin monitoring module is configured to attach at the mirror back plate via a plurality of fasteners.

16. The vehicular cabin monitoring system of claim 1, wherein an interior cavity of the cabin monitoring module is formed at least in part by the housing and the cover element attached at the housing, and wherein the camera and the near infrared light emitter are accommodated within the interior cavity of the cabin monitoring module.

17. The vehicular cabin monitoring system of claim 16, wherein the cabin monitoring module is disposed at least partially within a mirror casing of the mirror head.

18. The vehicular cabin monitoring system of claim 1, wherein the cutout region is formed at a lower edge of the mirror reflective element, and wherein an outer surface of the cover element is coplanar with an outer surface of the mirror reflective element at the lower edge of the mirror reflective element.

19. The vehicular cabin monitoring system of claim 1, wherein the cutout region is formed at a lower edge of the mirror reflective element, and wherein an outer surface of the cover element is at an oblique angle relative to an outer surface of the mirror reflective element at the lower edge of the mirror reflective element.

20. A vehicular cabin monitoring system, the vehicular cabin monitoring system comprising:
a vehicular interior rearview mirror assembly comprising a mirror head adjustable about a mounting structure, wherein the mounting structure is configured to mount the vehicular interior rearview mirror assembly at an interior portion of an interior cabin of a vehicle;
wherein the mirror head accommodates a mirror reflective element;
a cabin monitoring module attached at the mirror head, wherein the cabin monitoring module moves together and in tandem with the mirror head when, with the vehicular interior rearview mirror assembly mounted at the interior portion of the interior cabin of the vehicle, the mirror head is adjusted about the mounting structure to set a rearward view provided by the mirror reflective element for a driver of the vehicle;
wherein the cabin monitoring module comprises a housing that is disposed at least partially within the mirror head, and wherein the housing accommodates a camera, and wherein, with the vehicular interior rearview mirror assembly mounted at the interior portion of the interior cabin of the vehicle, and with the mirror head adjusted about the mounting structure to set the rearward view for the driver of the vehicle, the camera views at least (i) a driver's head region of the interior cabin of the vehicle and (ii) a passenger seating region of the interior cabin of the vehicle;
wherein the housing of the cabin monitoring module accommodates a near infrared light emitter that is electrically operable to emit near infrared light, and wherein the near infrared light emitter, when electrically operated to emit near infrared light, and with the vehicular interior rearview mirror assembly mounted at the interior portion of the vehicle, and with the mirror head adjusted about the mounting structure to set the rearward view for the driver of the vehicle, emits near infrared light at least toward (i) the driver's head region and (ii) the passenger seating region;
wherein the cabin monitoring module includes a cover element attached at the housing, and wherein, with the cabin monitoring module attached at the mirror head, the cover element is disposed at a cutout region of the mirror reflective element;
wherein an interior cavity of the cabin monitoring module is formed at least in part by the housing and the cover element attached at the housing, and wherein the camera and the near infrared light emitter are accommodated within the interior cavity of the cabin monitoring module;
an electronic control unit (ECU);
wherein image data captured by the camera is transferred to the ECU;
wherein the ECU comprises electronic circuitry and associated software, and wherein the electronic circuitry of the ECU comprises an image processor operable to process image data captured by the camera and transferred to the ECU; and
wherein, with the vehicular interior rearview mirror assembly mounted at the interior portion of the interior cabin of the vehicle, image data captured by the camera is processed at the ECU for (i) monitoring the driver of the vehicle and (ii) detecting a passenger present at the passenger seating region of the vehicle.

21. The vehicular cabin monitoring system of claim 20, wherein at least one selected from the group consisting of (i) a driver gaze direction is determined based on processing at the ECU of image data captured by the camera, (ii) driver attentiveness is determined based on processing at the ECU of image data captured by the camera, (iii) driver drowsiness is determined based on processing at the ECU of image data captured by the camera and (iv) driver distraction is determined based on processing at the ECU of image data captured by the camera.

22. The vehicular cabin monitoring system of claim 20, wherein the housing of the cabin monitoring module accommodates the ECU.

23. The vehicular cabin monitoring system of claim 20, wherein the cabin monitoring module comprises a self-contained unit that is attached at the mirror head and that electrically connects to a wire harness at the mirror head for connection to a power source of the vehicle when the vehicular interior rearview mirror assembly is mounted at the interior portion of the interior cabin of the vehicle.

24. The vehicular cabin monitoring system of claim 20, wherein the cutout region is formed at a lower edge of the mirror reflective element, and wherein an outer surface of the cover element is coplanar with an outer surface of the mirror reflective element at the lower edge of the mirror reflective element.

25. A vehicular cabin monitoring system, the vehicular cabin monitoring system comprising:
a vehicular interior rearview mirror assembly comprising a mirror head adjustable about a mounting structure, wherein the mounting structure is configured to mount the vehicular interior rearview mirror assembly at an interior portion of an interior cabin of a vehicle;
wherein the mirror head accommodates a mirror reflective element;
a cabin monitoring module attached at the mirror head, wherein the cabin monitoring module moves together and in tandem with the mirror head when, with the vehicular interior rearview mirror assembly mounted at the interior portion of the interior cabin of the vehicle, the mirror head is adjusted about the mounting structure to set a rearward view provided by the mirror reflective element for a driver of the vehicle;
wherein the cabin monitoring module comprises a housing that is disposed at least partially within the mirror head, and wherein the housing accommodates a camera, and wherein, with the vehicular interior rearview mirror assembly mounted at the interior portion of the interior cabin of the vehicle, and with the mirror head adjusted about the mounting structure to set the rearward view for the driver of the vehicle, the camera views at least a driver's head region of the interior cabin of the vehicle;
wherein the housing of the cabin monitoring module accommodates a near infrared light emitter that is electrically operable to emit near infrared light, and wherein the near infrared light emitter, when electrically operated to emit near infrared light, and with the vehicular interior rearview mirror assembly mounted at the interior portion of the vehicle, and with the mirror head adjusted about the mounting structure to set the rearward view for the driver of the vehicle, emits near infrared light at least toward the driver's head region;
wherein the cabin monitoring module includes a cover element attached at the housing, and wherein, with the cabin monitoring module attached at the mirror head, the cover element is disposed at a cutout region of the mirror reflective element;
wherein the cutout region is formed at a lower edge of the mirror reflective element, and wherein an outer surface of the cover element correspond to an outer surface of the mirror reflective element at the lower edge of the mirror reflective element;
wherein the cabin monitoring module comprises a self-contained unit that is attached at the mirror head and that electrically connects to a wire harness at the mirror head for connection to a power source of the vehicle when the vehicular interior rearview mirror assembly is mounted at the interior portion of the interior cabin of the vehicle;
an electronic control unit (ECU);
wherein image data captured by the camera is transferred to the ECU;
wherein the ECU comprises electronic circuitry and associated software, and wherein the electronic circuitry of the ECU comprises an image processor operable to process image data captured by the camera and transferred to the ECU;
wherein the housing of the cabin monitoring module accommodates the ECU; and
wherein, with the vehicular interior rearview mirror assembly mounted at the interior portion of the interior cabin of the vehicle, image data captured by the camera is processed at the ECU for monitoring the driver of the vehicle.

26. The vehicular cabin monitoring system of claim 25, wherein image data captured by the camera is processed at the ECU for at least one selected from the group consisting of (i) detecting a passenger of the vehicle and (ii) monitoring a passenger of the vehicle.

27. The vehicular cabin monitoring system of claim 25, wherein the near infrared light emitter, when electrically operated to emit near infrared light, and with the mirror head adjusted about the mounting structure to set the rearward view for the driver of the vehicle, emits near infrared light to illuminate a passenger seating region of the vehicle.

28. The vehicular cabin monitoring system of claim 25, wherein at least one selected from the group consisting of (i) a driver gaze direction is determined based on processing at the ECU of image data captured by the camera, (ii) driver attentiveness is determined based on processing at the ECU of image data captured by the camera, (iii) driver drowsiness is determined based on processing at the ECU of image data captured by the camera and (iv) driver distraction is determined based on processing at the ECU of image data captured by the camera.

29. The vehicular cabin monitoring system of claim 25, wherein the housing of the cabin monitoring module is configured to attach at a mirror back plate that adhesively attaches at the mirror reflective element.

30. The vehicular cabin monitoring system of claim 25, wherein the outer surface of the cover element is coplanar with the outer surface of the mirror reflective element at the lower edge of the mirror reflective element.

31. The vehicular cabin monitoring system of claim 25, wherein the outer surface of the cover element is at an oblique angle relative to the outer surface of the mirror reflective element at the lower edge of the mirror reflective element.
